(19) United States
(12) Patent Application Publication
(10) Pub. No.: US 2020/0164131 A1
(43) Pub. Date: May 28, 2020

(54) METHOD AND APPARATUS FOR GENERATING TREATMENT REGIMENS

(71) Applicant: Fresenius Medical Care Deutschland GmbH, Bad Homburg (DE)

(72) Inventors: Gheorghe MUSCA, Bucharest (RO); Ariana POPESCU, Bucharest (RO)

(21) Appl. No.: 16/613,483

(22) PCT Filed: Jun. 8, 2018
(86) PCT No.: PCT/EP2018/065163

§ 371 Date: Nov. 14, 2019

(30) Foreign Application Priority Data
Jun. 12, 2017 (RO) A 2017 00362

(51) Int. Cl. A61M 1/28; G16H 10/60; G16H 20/40; G16H 50/50; G16H 40/60

(52) U.S. Cl. CPC A61M 1/28; G16H 10/60; A61M 2205/52; G16H 50/50; G16H 40/60; G16H 20/40

(57) ABSTRACT

A method for generating treatment regimens includes: receiving a therapy target and a therapy restriction input; receiving a patient characteristics input; generating a plurality of peritoneal dialysis treatment regimens, each peritoneal dialysis treatment regimen being defined by a succession of first exchange cycles and second exchange cycles and by a corresponding number of first exchange cycles and a number of second exchange cycles; simulating a peritoneal dialysis treatment outcome for each of the plurality of peritoneal dialysis treatment regimens based at least in part on the patient characteristics to generate a simulated treatment outcome; and providing for selection a peritoneal dialysis treatment regimen to be applied for a specific treatment based on the simulated treatment outcome and the therapy target and restrictions.

### CROSS-REFERENCE TO RELATED APPLICATIONS

The present application is the national stage entry of International Patent Application No. PCT/EP2018/065163, filed on Jun. 8, 2018, and claims priority to Application No. 201700362, filed in Romania on Jun. 12, 2017, the disclosure of which are expressly incorporated herein in entirety by reference thereto.

### TECHNICAL FIELD

The present disclosure relates to the field of generating a dialysis treatment regimen. The present disclosure further relates to generating a regimen for operating a peritoneal dialysis treatment apparatus.

### BACKGROUND

The proportion of patients performing automated peritoneal dialysis (“APD”) is increasing worldwide, which is due at least in part to the ability of APD to be adapted to a patient's particular needs.

The two primary goals of dialysis, solute clearance and ultrafiltration (“UF”), depend on the modality or type of APD performed. Historically, APD devices did not have the capability to provide feedback to the patient regarding the effectiveness of the patient's recent therapies. Also, APD devices would typically run open loop such that they did not adjust therapy parameters (e.g., modality, solution type, therapy time and fill volume) based on the actual measured daily clearance and UF. Accordingly, a substantial risk existed that some patients would underachieve their targets and develop adverse conditions, such as fluid overload and hypertension.

To address the above problems, computerised prescription optimization systems were developed that use patient physiological data in combination with specified therapy targets (e.g., ultrafiltration volume, clearance) and ranges of therapy inputs (e.g., number of exchanges, inflow volumes, cycle times, solution choices) to calculate or deliver possible regimens that fit within the provided ranges. Patient characteristics may include mass transfer area coefficient (“MTAC”) data and hydraulic permeability data to classify the patient's transport and UF characteristics. In order to simulate the outcome of a peritoneal dialysis treatment, various algorithms have been developed and described in the literature [Kinetic modelling in Peritoneal Dialysis, Gotch, Keen, Clinical Dialysis, 4th edition, 2005: 385-419]. One algorithm frequently used to simulate a dialysis treatment outcome employs a three-pore model, which adds predicted clearances across three different sized pores in the patient's peritoneal membrane. Flow of toxins through large pores, small pores and micropores in the blood vessels of the peritoneal membrane are summed. Urea and creatinine for example are removed from the patient's blood through the small pores.

The large pores allow larger protein molecules to pass from the patient's blood to the dialysate. In each case, an osmotic gradient drives the toxins from the patient's blood to the dialysate. In a more simplified two pore model only two different sized pores are considered. Alternatively a so called two-pool peritoneal dialysis kinetic model can be used to predict fluid and solute removal in peritoneal dialysis to simulate a therapy outcome. The two pool model utilizes a set of differential equations that collectively describe both diffusive and convective mass transport in both the body and dialysate compartments for an “equivalent” membrane core.

For example, a prescription optimization system typically applies a two pore, three pore or two pool model or other model to use target information and other therapy input information to generate the regimens.

However, the above-described conventional dialysis therapy regimens do not always provide optimum treatment outcomes, resulting in patients underachieving their therapy objectives.

### SUMMARY

This document describes an improved method, as well as corresponding devices and systems, for generating a peritoneal dialysis treatment regimen for controlling peritoneal dialysis treatment for improved achievement of therapy objectives. Specifically, this document describes methods of generating a peritoneal dialysis treatment regimen for controlling the operation of a peritoneal dialysis treatment machine to meet a therapy target or therapy target range.

In a first general aspect, a method for generating a peritoneal dialysis regimen includes: receiving a therapy target input; receiving a patient characteristics input; generating a plurality of peritoneal dialysis treatment regimens, each peritoneal dialysis regimen being defined by a succession of first exchange cycles and second exchange cycles and by a number of first exchange cycles and a number of second exchange cycles; simulating a peritoneal dialysis treatment outcome for each of the plurality of peritoneal dialysis treatment regimens based at least in part on the patient characteristics to generate a simulated treatment outcome; and providing for selection a peritoneal dialysis treatment regimen to be applied for a specific treatment based on the simulated treatment outcome and the therapy target.

Embodiments disclosed herein may comprise one or several of the following features.

In some embodiments described herein, the first exchange cycles are defined by a first inflow volume and a first dwell time, and the second exchange cycles are defined by a second inflow volume and a second dwell time.

In some embodiments described herein, the number of first exchange cycles and/or the number of second exchange cycles are variable and/or independent from each other.

In some embodiments described herein, the method includes receiving a treatment parameter range input, and describing an allowable range for one or more treatment parameters, wherein the plurality of peritoneal dialysis regimes are generated within the allowable range of the one or more treatment parameters.

In some embodiments described herein, a first glucose concentration is defined for the first exchange cycles, and a second glucose concentration is defined for the second exchange cycles, the second concentration being different from the first concentration.

In another general aspect, a method of generating a peritoneal dialysis regimen for controlling the operation of a peritoneal dialysis treatment machine to meet a therapy target or therapy target range, may include: receiving a therapy target input; receiving a patient characteristics input; generating a plurality of peritoneal dialysis treatment regimens, each peritoneal dialysis treatment regimen being defined by a succession of first exchange cycles and second exchange cycles, wherein the first exchange cycles are defined by a first inflow volume and a first dwell time, and the second exchange cycles are defined by a second inflow volume and a second dwell time, wherein the first exchange cycles are shorter and smaller than the second exchanges cycles, and wherein a first glucose concentration is defined for the first exchange cycles and a second glucose concentration is defined for the second exchange cycles, wherein the first glucose concentration is larger than the second glucose concentration; simulating a peritoneal dialysis treatment outcome for each of the plurality of peritoneal dialysis treatment regimens based at least in part on the patient characteristics to generate a simulated treatment outcome; and providing for selection a peritoneal dialysis treatment regimen to be applied for a specific treatment based on the simulated treatment outcome and the inputted therapy target.

Embodiments disclosed herein may comprise one or several of the following features.

In some embodiments described herein, the method includes receiving an allowable treatment parameter range, wherein the plurality of peritoneal dialysis treatment regimens are generated based at least in part on the allowable treatment parameter range.

In some embodiments described herein, a fixed ratio between first dwell time and second dwell time is predefined.

In some embodiments described herein, a fixed ratio between first inflow volume and second inflow volume is predefined.

In some embodiments described herein, the first exchange cycles precede the second exchange cycles.

In some embodiments described herein, the first exchange cycles and the second exchange cycles alternate.

In another general aspect, a device for generating a peritoneal dialysis treatment regimen for controlling the operation of a peritoneal dialysis treatment machine to meet a therapy target or a therapy target range includes: a therapy target input unit; a patient characteristics input unit; a peritoneal regimen generation unit configured to generate a plurality of peritoneal dialysis treatment regimens, each peritoneal dialysis treatment regimen being defined by a succession of first exchange cycles and second exchange cycles, the first exchange cycles being defined by a first inflow volume and a first dwell time, and the second exchange cycles being defined by a second inflow volume and a second dwell time, wherein the first exchange cycles are shorter and smaller than the second exchange cycles, and wherein a first glucose concentration is defined for the first exchange cycles and a second glucose concentration is defined for the second exchange cycles, wherein the first glucose concentration is larger than the second concentration; a simulation unit configured to simulate a peritoneal dialysis treatment outcome for each of the plurality of peritoneal dialysis treatment regimens to generate a simulated treatment outcome; and a selection unit configured to facilitate selection of a peritoneal dialysis treatment regimen to be applied for a specific treatment from the plurality of peritoneal dialysis treatment regimens based on the respective simulated treatment outcome and the therapy target.

Embodiments disclosed herein may comprise one or several of the following features.

In some embodiments described herein, the apparatus is communicably coupled to a peritoneal dialysis machine.

In some embodiments described herein, the apparatus is communicably coupled to a solution mixing unit for mixing a peritoneal dialysis solution from input solutions to create a peritoneal dialysis solution of the first glucose concentration and a peritoneal dialysis solution of the second glucose concentration, a control unit adapted to control the mixing unit, and/or an infusion unit to operate in accordance with the selected treatment regimen.

In some embodiments described herein, the apparatus is communicably coupled to a database module of a database.

In another general aspect, a peritoneal dialysis system includes a database with a database module; and a peritoneal dialysis machine connected to the database for downloading a peritoneal dialysis regimen from the database and adapted to operate in accordance with the downloaded peritoneal dialysis regimen.

In some embodiments described herein, the peritoneal dialysis regimen received from the database is selected based on a simulated treatment outcome and a therapy target.

In another general aspect, a computer program includes instructions which, when being executed by a computer, cause the computer to execute the methods described above.

In another general aspect, computer-readable medium include instructions for the execution of the methods described above.

### BRIEF DESCRIPTION OF THE FIGURES

FIG. 1 depicts a method of generating a peritoneal dialysis treatment regimen and applying the generated treatment regimen to a patient.

FIG. 2 depicts a peritoneal dialysis treatment regimen generation device.

FIG. 3 depicts a peritoneal dialysis machine including a treatment regimen generation device.

FIG. 4 depicts a peritoneal dialysis system including a database and a peritoneal dialysis machine.

FIGS. 5-10 depict graphical representations of candidate sets of peritoneal dialysis treatment regimens.

### DETAILED DESCRIPTION OF THE FIGURES

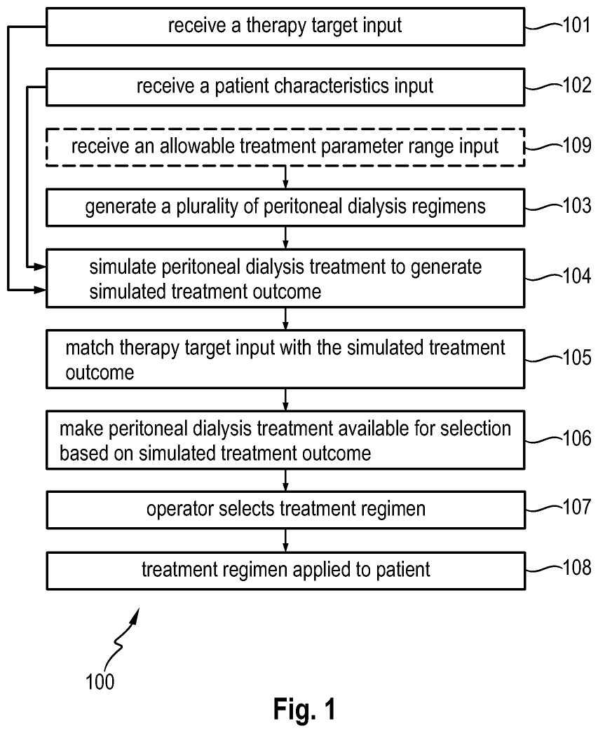

FIG. 1 depicts a method 100 of generating a peritoneal regimen for controlling the operation of a peritoneal dialysis treatment machine to perform a peritoneal dialysis treatment. Each peritoneal dialysis regimen generated by the method 100 depicted in FIG. 1 defines a peritoneal dialysis treatment in terms of the peritoneal dialysis solution applied during the treatment (and thereby the glucose concentration), as well as by the number, fill volume, and timing of the sequence of exchange cycles, including a filling time, a dwell time and a drain time.

The method 100 comprises an inputting step 101 of receiving a therapy target input, or therapy target range input, in particular a peritoneal dialysis therapy target or target range input from a patient database or from an operator, such as a patient, nurse, physician, caregiver, or other person assisting the patient in supervising his or her therapy. A therapy target value defines a desired therapy outcome in terms of one or more parameter values describing the therapy outcome. The therapy target value may be prescribed by a physician. In one embodiment, a therapy target is defined by an ultrafiltration volume, by a solute clearance, such as a creatinine or urea clearance, or by a combination of the above.

Advantageously, the method 100 comprises an inputting step 109 of receiving a treatment parameter range input from a database or from an operator. The treatment parameter range describes an allowable range for one or more treatment parameters (i.e., a set of one or more treatment restrictions). The treatment parameter range may be associated with a particular patient, a particular peritoneal dialysis machine, or a particular peritoneal dialysis machine type. For example, a therapy restriction may define a certain safety parameter, such as a maximum inflow volume allowed for a specific patient, and/or a certain quality of life specific parameter, such as a maximum total therapy time. For example, APD patients may be required to complete night APD treatment by a certain defined time, and therefore the total therapy time has to be limited.

Still referring to FIG. 1, the method 100 includes a further inputting step 102 of receiving a patient characteristics input, such as a peritoneum transport characteristics, body surface area (BSA), or other patient characteristics. The patient characteristics input may be received from a database or from an operator. A body surface area of the patient received at step 102 may be estimated based on an epidemiological equation, such as the Du Bois equation, using the height and the weight of the patient as input. The peritoneum transport characteristics of the patient received at step 102, which are sometimes termed as transporter type, may be determined by a peritoneal function test. For certain predefined equilibrium conditions, a particular dialysate over plasma ratio (D/P ratio) for creatinine, or possibly urea, may be regarded as corresponding to a particular transporter type.

In regimen generation step 103, a set of peritoneal dialysis treatment regimens within a parameter range or “solution space” (e.g. within the previously inputted allowable treatment parameter range/therapy restriction(s)) is generated. In some implementations, each peritoneal dialysis treatment regimen generated by step 103 is defined by a succession of exchange cycles, which in turn are defined by inflow volume (i.e., the volume of peritoneal dialysis solution initially infused into the patient), dwell time (i.e., the time during which solution is neither actively withdrawn or infused), and total exchange time (i.e., the sum of infusion time, dwell time, and withdrawal or drain time). Furthermore a peritoneal dialysis treatment regimen may also include a description of the peritoneal dialysis solution to be applied for a certain treatment, including a value describing the glucose concentration of the solution.

In some implementations, each peritoneal dialysis treatment regimen is defined by a succession of first exchange cycles and second exchange cycles, the first exchange cycles of each of the plurality of peritoneal dialysis treatment regimens being defined by a first inflow volume and a first dwell time, and the second exchange cycles of each of the plurality of peritoneal dialysis treatment regimens being defined by a second inflow volume and a second dwell time. Each treatment regimen is defined by a number of first exchange cycles and by a number of second cycles, the number of first exchange cycles and/or the number of second exchange cycles is variable and/or independent from each other. In some embodiments, the first exchange cycles are shorter and smaller than the second exchange cycles.

The inflow volume of the first (i.e. smaller) exchange cycles may be determined according to a first predefined relation to the body surface area, such as 800 ml/m\hat{ }2 BSA of the patient. The dwell time and total cycle time of the shorter exchange cycles may each be determined according to a corresponding predefined relation to the transporter type or peritoneum transport characteristics for the shorter exchange cycles. The inflow volume of the second (i.e. larger) exchange cycles may be determined according to a second predefined relation to the body surface area of the patient, such as 1500 ml/m\hat{ }2 BSA of the patient. The dwell time and total cycle time of the longer exchange cycles may each be determined according to a corresponding predefined relation to the peritoneum transport characteristics or transporter type for the longer exchange cycles.

In some implementations, the shorter and smaller exchange cycles precede the longer and larger exchange cycles, and the number of shorter and smaller/shorter exchange cycles is a first degree of freedom and the number of larger/longer exchange cycles is a further degree of freedom within the solution space or parameter range for peritoneal dialysis treatment regimens. In some embodiments, the number of first exchange cycles is independent from the number of second exchange cycles, and both the number of first exchange cycles and the number of second exchange cycles are variable.

Another degree of freedom within the parameter range or solution space of peritoneal dialysis treatment regimens may be the peritoneal dialysis solution type to be applied and/or glucose concentration of the peritoneal dialysis solution to be applied. The parameter range of the glucose concentration may be sampled depending on the demands of the subsequent simulation step and/or depending on available peritoneal dialysis solutions, as well as based on the capabilities of the peritoneal dialysis (PD) machine. In some embodiments, the PD machine may be provided with the capability to provide glucose profiling (i.e. the capability to adapt the glucose concentration during treatment). A first glucose concentration may be defined for the first exchange cycles, and a second glucose concentration may be defined for the second exchange cycles. In some embodiments, the solution type and/or glucose concentration of the peritoneal dialysis solution to be applied may be predefined.

In some embodiments, the regimen generation step 103 involves the use of a fixed ratio defined between the size of the exchange volumes of small (i.e., first) exchange cycles and the size of the exchange volumes of large (i.e., second) exchange cycles. In such implementations, the absolute volume of either the small exchange cycle or the large exchange cycle is an additional degree of freedom. Thus, the parameter range or solution space may have the following degrees of freedom: the absolute size of either the small exchange cycles or the large exchange cycles, the number of small exchange cycles, the number of large exchange cycles, and the solution type/glucose concentration of the peritoneal dialysis solution, or the first and second glucose concentrations of the peritoneal dialysis solution described above. The parameter range may be sampled depending on the demands of the subsequent simulation step and/or, in the case of the glucose concentration, depending on available peritoneal dialysis solutions and/or PD machine capabilities.

In some implementations, the regimen generation step 103 includes calculating the dwell times and total cycle times for the shorter and for the longer exchange cycles according to a respective predefined relation to the transport characteristics or transporter type. In such embodiments, the absolute volume of the small (i.e., first) exchange cycle and the large (i.e., second) exchange cycle are additional degrees of freedom. Thus, the parameter range or solution space may have the following degrees of freedom: the absolute size of the small exchange cycles and the large exchange cycles, the number of small exchange cycles, the number of large exchange cycles, and the solution type/glucose concentration of the peritoneal dialysis solution, or the first and the second glucose concentrations of the peritoneal dialysis solution described above. The parameter range may be sampled according to the demands of the subsequent simulation step, and/or available peritoneal dialysis solutions, and/or PD machine capabilities (for the glucose concentration).

In some embodiments, the dwell times of the larger (i.e., second) and the smaller (i.e., first) exchange cycles are additional degrees of freedom. Furthermore, in some embodiments, the absolute volumes of the small exchange cycles and the large exchange cycles are variable. Thus, the solution space or parameter range may have the following degrees of freedom: the dwell time of the small exchange cycles and of the large exchange cycles, the absolute size of the small exchange cycles and the large exchange cycle, the number of small exchange cycles, the number of large exchange cycles, and the glucose concentration of the peritoneal dialysis solution, or the first and the second glucose concentrations of the peritoneal dialysis solution described above.

Still referring to FIG. 1, in a simulation step 104, the prescription parameters are varied within the predefined (allowable) treatment parameter range or solution space, and the prescription parameters are input into a simulation tool to determine a simulated treatment outcome or quality parameter. In some embodiments, the simulated treatment outcome is expressed in terms of ultrafiltration, creatinine or urea clearance, or in terms of a cost function combining these parameters. For the simulation of the treatment outcome a three pore model, a two pore model, or a two pool model may be applied.

In a matching step 105, the previously input therapy target or therapy target range is matched with the simulated treatment outcome for each of the peritoneal dialysis treatment regimens within the set of peritoneal dialysis treatment regimens to generate a set of prescription candidates that meet the predefined allowable treatment target or treatment target range, or in other words, the prescriptions of the set of prescription candidates within the allowable treatment parameter range having a simulated treatment outcome within the allowable treatment outcome range.

The prescriptions within the set of treatment regimen candidates are made available for selection by an operator in a step 106. The operator may then select a prescription to be applied for a certain treatment within the set of selectable peritoneal dialysis treatment regimen in step 107. The prescription can also further be optimized manually.

Finally, in step 108, a peritoneal dialysis treatment in accordance with the selected peritoneal dialysis treatment regimen is performed on the patient. To perform the selected peritoneal dialysis treatment regimen, the machine is supplied with one or more peritoneal dialysis solution bags containing the peritoneal dialysis solution or solutions for the particular selected treatment regimen. For example, the machine is supplied with a peritoneal dialysis solution having the appropriate solution type and glucose concentration, or the appropriate solution(s) and concentrations(s) that correspond(s) to the selected treatment regimen are mixed and provided to the machine. The patient is connected to the peritoneal dialysis machine and the peritoneal dialysis machine is operated to apply a succession of treatment cycles in accordance with the selected treatment regimen. For example, the pumps and valves of the machine are operated to supply and withdraw peritoneal dialysis fluid at the defined inflow volume of the respective exchange cycles, and maintain the glucose concentration of the respective exchange cycles, as well as the total cycle time and dwell time as defined by the schedule of first and second exchange cycles of the treatment regimen.

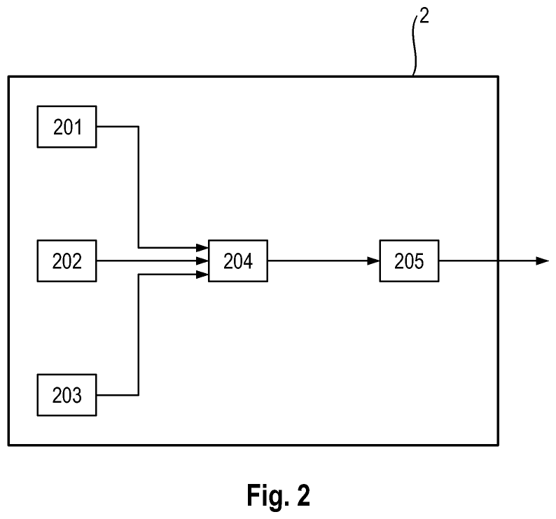

FIG. 2 depicts a peritoneal dialysis treatment regimen generating device 2 for generating a peritoneal dialysis treatment regimen for controlling the operation of a peritoneal dialysis machine to operate to apply a succession of first exchange cycles and second exchange cycles within an allowable treatment parameter range and/or therapy restriction(s), and thereby meet a therapy target or therapy target range. The regimen generating device 2 is adapted to be operated by applying the method 100 described above with reference to FIG. 1.

The regimen generation device 2 includes a therapy target input unit 201 for inputting a therapy target or therapy target range for a particular patient. The therapy target may be input using a manually operated I/O device or received from a database administrating patient data records that includes a therapy target or therapy target range as part of each patient data record. The therapy target or therapy target range may be prescribed by a physician.

The regimen generation device 2 further includes a patient characteristics input unit 202 for inputting patient characteristics. The patient characteristics may be input using a manually operated I/O device, or may be received from a database administrating patient data records that includes patient characteristics as part of each patient data record. Patient characteristics may include peritoneum transport characteristics and other patient characteristics as described above in relation to the method 100 of FIG. 1.

The regimen generation device 2 further includes a peritoneal dialysis regimen generation unit 203 configured to generate an initial set of peritoneal dialysis regimens within the allowable treatment parameter range. Each peritoneal dialysis regimen within the initial set of peritoneal dialysis treatment regimens is defined by a succession of first exchange cycles and second exchange cycles, the first exchange cycles being defined by a first inflow volume, a first dwell time and a first glucose concentration, and the second exchange cycles being defined by a second inflow volume, a second dwell time, and a second glucose concentration. In some implementations, each treatment regimen within the initial set of peritoneal dialysis treatment regimens is defined by a number of first exchange cycles and a number of second exchange cycles, with the number of first exchange cycles and/or the number of second exchange cycles being variable and/or independent from each other.

The peritoneal dialysis treatment regimen generation unit 203 may be configured to receive an allowable treatment parameter range input, wherein the treatment parameter range defines an interval of treatment parameters for the set of peritoneal dialysis treatment regimes. The allowable treatment parameter range and/or therapy restriction(s) may be included in a patient data record associated with the patient to be treated. The allowable treatment parameter range and/or therapy restriction(s) may be prescribed by a physician.

As depicted in FIG. 2, the therapy target input unit 201, the patient characteristics input unit 202, and the peritoneal dialysis regimen generation unit 203 are each connected to a simulation unit 204. The simulation unit 204 is configured to simulate a peritoneal dialysis treatment outcome for each of the plurality of peritoneal dialysis treatment regimens to generate a simulated treatment outcome.

The simulation unit 204 is further adapted to match the simulated treatment outcome and the therapy target or therapy target range for the peritoneal dialysis treatment regimens within the set of peritoneal dialysis treatment regimens (i.e. the allowable treatment parameter range and/or therapy restriction(s)) in order to generate or determine a set of treatment regimen candidates that meet the predefined allowable treatment target or treatment target range.

The simulation unit 204 is connected to the selection unit 205 for transmitting the set of peritoneal dialysis treatment regimen candidates to the selection unit 205. The selection unit 205 provides the peritoneal dialysis treatment candidates to an operator for selection of the peritoneal dialysis treatment regimen to be applied for a specific treatment.

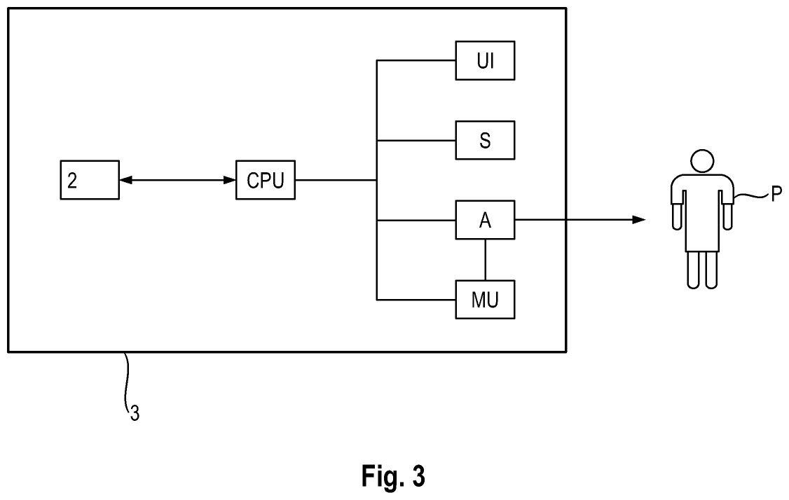

FIG. 3 depicts an embodiment of a peritoneal dialysis machine 3 that includes the regimen generation device 2 of FIG. 2. As depicted in FIG. 3, the peritoneal dialysis machine 3 also includes a user interface UI, at least one sensor S, and an infusion unit A, which includes one or more actors, such as valves and pumps that operate the peritoneal dialysis machine in accordance with a particular treatment regimen. The infusion unit A is fluidly connected to the patient P receiving peritoneal dialysis treatment to supply peritoneal dialysis solution to the patient P and to withdraw a solution comprising metabolic waste from the patient P. A certain solution concentration may be defined for each of the cycles of the treatment regimen. In some embodiments, a dialysis solution with a specified concentration of glucose is applied for each cycle of the treatment regimen. In some implementations, the concentration of glucose of the dialysis solution is selected from a predefined set of concentrations (e.g. 1.5%, 2.3% or 4.25% glucose). Solution sources, such as solution bags with specified solution concentrations (e.g. of 1.5%, 2.3% or 4.25% glucose) may be sequentially connected to the infusion unit A to infuse a dialysis solution of the appropriate concentration defined for a certain exchange cycle of a treatment regimen to a patient.

In some embodiments, the infusion unit A is connected to a solution mixing unit MU for mixing a peritoneal dialysis solution from input solutions to create one or more concentrations of peritoneal dialysis solution required for the peritoneal dialysis treatment regimen. The solution mixing unit MU is connected to at least two solution sources (input concentrates), such as solution bags of defined solution concentrations (e.g. of 1.5%, 2.3% or 4.25% glucose). The mixing of input concentrates may be performed within the solution mixing unit MU. Alternatively, the mixing of input concentrates may be performed within the peritoneal abdomen of the patient by sequentially infusing a solution of a first concentration and a solution of a second concentration into the patient's abdomen, the sequential infusing being controlled by the solution mixing unit. If sequential infusion and mixing within the patient's abdomen is applied, the inflow phase may be divided into two distinct inflow phases, each with a different solution (glucose) concentration. In some embodiments, the glucose concentration for a cycle may be varied within an interval e.g. [1.5%, 2.3%] or [2.3%, 4.25%] glucose, depending on the concentrations available for the solution sources. For example, a cycle with volume V and glucose concentration g (e.g., g having any value between 1.5% and 2.3% glucose) may be defined within a treatment regimen. Two adjacent inflow phases are determined with volumes V1 and V2, and, for example, glucose concentrations g1=1.5% and g2=2.3% (or g1=2.3% and g2=4.25%), so that:

V1+V2=V

g1*V1+g2*V2=g*V

Given g1, g2, g and V, the equations above can be solved to compute volumes V1 and V2. The solution mixing unit MU may be controlled to perform a sequence of two inflows (V1, g1) and (V2, g2) that will result in a volume V and an equivalent mixed glucose g within the patient's abdomen. This mixing of concentrates may be done for each exchange cycle of the treatment regimen, with the only restriction being not to exceed the maximum number of bags that the solution mixing unit may handle (e.g. 6 bags).

As depicted in FIG. 3, the user interface UI, the sensor S, the infusion unit A, and the regimen generation device 2 are each connected to a central processing unit CPU. The central processing unit CPU receives the selected peritoneal dialysis treatment regimen for a particular treatment from the regimen generation device 2 and controls the infusion unit A and/or the solution mixing unit MU to perform a peritoneal dialysis treatment on patient P by applying the fluid exchange cycles in accordance with the selected peritoneal dialysis treatment regimen.

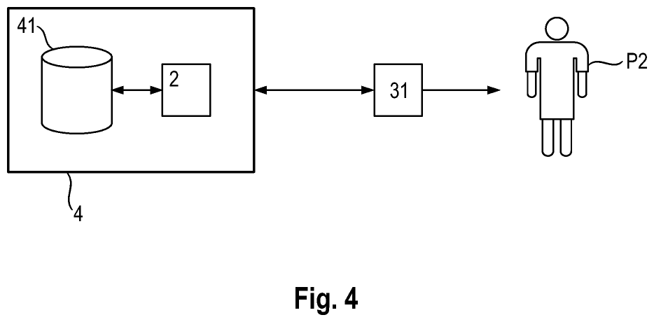

FIG. 4 depicts a peritoneal dialysis system comprising a patient database 4 for supplying a treatment regimen or a set of treatment regimens to a peritoneal dialysis device 31. As depicted in FIG. 4, peritoneal dialysis machine 31 is fluidly connected to a patient P2 for applying a peritoneal dialysis treatment to the patient P2. The patient database 4 comprises a database module 41 for administrating patient data records of peritoneal dialysis patients. Each patient data record may include patient characteristics, an allowable treatment parameter range and/or therapy restriction(s), and a therapy target or a therapy target range, as described above. The database module 41 is communicably connected to the regimen generation device 2 to provide patient characteristics and a therapy target, or therapy target range, to the regimen generation device 2 as described above in relation to FIG. 2. The patient database 4 is connected to the peritoneal dialysis machine 31 for transmitting a peritoneal dialysis treatment regimen to the machine 31. The peritoneal dialysis machine 31 is fluidly connected to the patient P2 to perform the peritoneal treatment in accordance with the received dialysis treatment regimen, as described above in relation to FIG. 3.

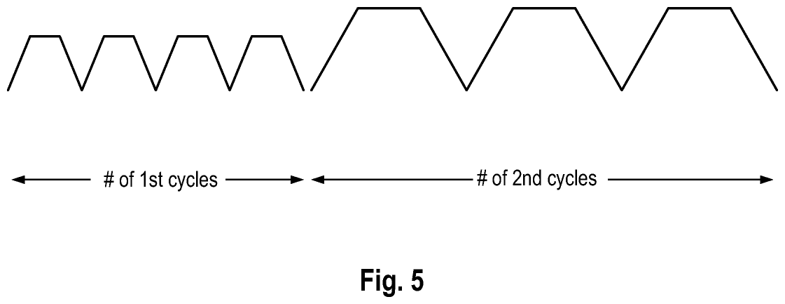

FIG. 5 is a graphical representation of a candidate set of peritoneal dialysis treatment regimens input into the simulation as described above. As depicted in FIG. 5, a number of first exchange cycles precedes a number of second exchange cycles, and the first exchange cycles are smaller and shorter and the second exchange cycles are larger and longer. Typically the shorter and smaller exchange cycles target ultrafiltration, whereas the longer and larger exchange cycles target solute clearance. In some implementations, a first glucose concentration is defined for the exchange cycles targeting ultrafiltration and a second glucose concentration is defined for the exchange cycles targeting solute clearance, and a larger glucose concentration is defined for the exchange cycles targeting ultrafiltration, thereby creating a larger osmotic gradient and/or osmotic pressure. In the embodiment depicted in FIG. 5, the number of first, (i.e. smaller and shorter) exchange cycles and the number of second exchange cycles are variable and independent from each other.

In the embodiment depicted in FIG. 5, the peritoneal dialysis treatment regimens have the following degrees of freedom: the number of first shorter and smaller exchange cycles, the number of second longer and larger exchange cycles, and the solution type and/or glucose concentration(s) of the peritoneal dialysis solution(s) to be applied for the treatment. The parameter range of the glucose concentration may be sampled depending on the demands of the subsequent simulation step and/or depending on available peritoneal dialysis solutions or PD machine capabilities.

In the embodiment of FIG. 5, inflow volume of each of the first and second exchange cycles is determined according to a first and a second predefined relation to the body surface area, respectively. The dwell time and total cycle time of each of the first and second exchange cycles may be determined according to a corresponding predefined relation to the transporter type or peritoneum transport characteristics for the respective first or second exchange cycles.

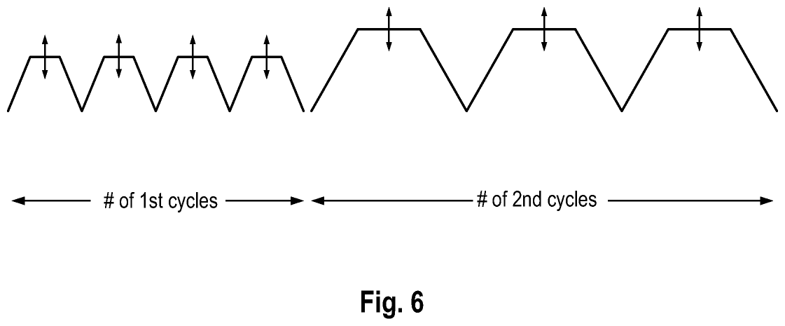

FIG. 6 is a graphical representation of an example candidate set of peritoneal dialysis treatments. As in FIG. 5, shorter and smaller exchange cycles in the candidate set of treatments in FIG. 6 precede larger and longer exchange cycles, and the number of first exchange cycles (i.e. smaller and shorter exchange cycles) and the number of second exchange cycles (i.e. larger and longer exchange cycles) are variable and independent from each other. Furthermore, the dwell time and total cycle time of the first exchange cycles and second exchange cycles may be each determined according to a corresponding predefined relation to the transporter type or peritoneum transport characteristics for the respective first or second exchange cycles. Unlike the embodiment depicted in FIG. 5, the absolute volume of the small and/or the large exchange cycles in the embodiment depicted in FIG. 6 is an additional degree of freedom. A fixed ratio may be defined between the size of the exchange volume of the small exchange cycles and the size of the exchange volume of the large exchange cycles. Alternatively, the exchange volumes of the large exchange cycles and the small exchange cycles may be varied independently. Thus, the parameter range or solution space has the following degrees of freedom: the volume of the small exchange cycles and/or the large exchange cycles, the number of small exchange cycles, the number of large exchange cycles, and the solution type and/or glucose concentration(s) of the peritoneal dialysis solution(s). The parameter range may be sampled according to simulation requirements and/or solution type availability, or mixing and/or solution profiling capabilities of the PD machine (for the glucose concentration).

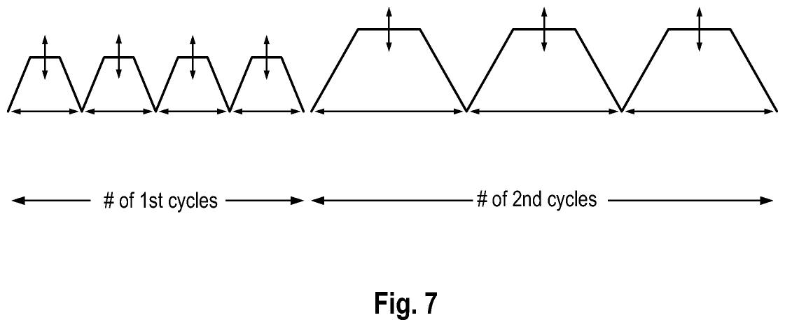

FIG. 7 is a graphical representation of an example candidate set of peritoneal dialysis treatments. As with the embodiments depicted in FIGS. 5 and 6, the treatment regime of FIG. 7 includes shorter and smaller exchange cycles preceding larger and longer exchange cycles, and the number of first exchange cycles (i.e., the smaller and shorter exchange cycles) and the number of second exchange cycles (i.e., the larger and longer exchange cycles) are variable and independent from each other.

Unlike the embodiments depicted in FIGS. 5, and 6, the dwell times of the larger exchange cycles and/or the smaller exchange cycles are additional degrees of freedom in the treatment regime of FIG. 7. A fixed ratio may be defined between the dwell time of the short exchange cycles and the dwell time of the long exchange cycles. Alternatively, the dwell times of the short exchange cycles and the long exchange cycles may be varied independently. Thus, the parameter range or solution space in the treatment regime of FIG. 7 has the following degrees of freedom: the absolute volume of the small exchange cycles and/or the large exchange cycles, the dwell time of the short exchange cycles and/or the long exchange cycles, the number of small exchange cycles, the number of large exchange cycles, and the solution type and/or glucose concentration(s) of the peritoneal dialysis solution(s). The parameter range may be sampled according to the demands of the subsequent simulation step, and/or available peritoneal dialysis solutions, or mixing and/or solution profiling capabilities of the PD machine (for the glucose concentration).

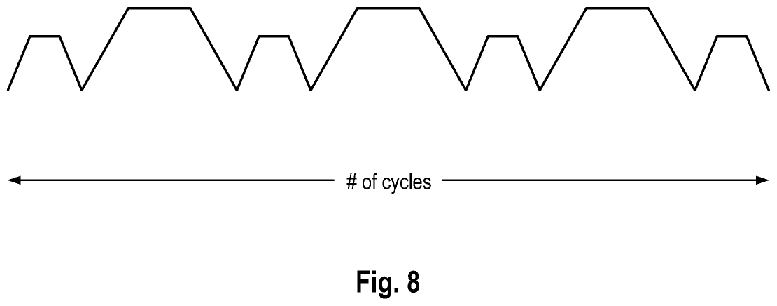

FIG. 8 is a graphical representation of an example candidate set of peritoneal dialysis treatment regimens input into the peritoneal dialysis treatment simulation. As depicted in FIG. 8, smaller and shorter exchange cycles of a first type alternate with larger and longer exchange cycles of a second type. Therefore, the number of shorter/smaller exchange cycles and the number of larger/longer exchange cycles are coupled. The number of first exchange cycles (i.e. smaller and shorter exchange cycles) and the number of second exchange cycles (i.e. larger and longer exchange cycles) of the treatment regimen depicted in FIG. 8 are variable.

In relation to the parameters of fill volume, dwell time and glucose concentration as parameters or degrees of freedom of a peritoneal dialysis treatment regimen, the candidate set of peritoneal dialysis treatment regimens depicted in FIG. 8 corresponds to the candidate set depicted in FIG. 5.

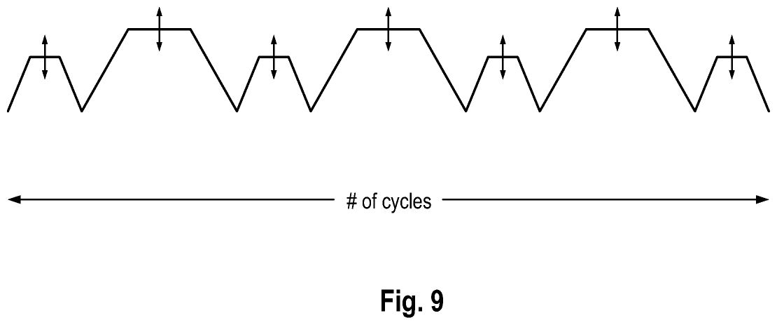

FIG. 9 is a graphical representation of another example candidate set of peritoneal dialysis treatment regimens input into the peritoneal dialysis treatment simulation. Similar to the candidate sets depicted in FIG. 8, smaller and shorter exchange cycles of a first type alternate with larger and longer exchange cycles of a second type, and the number of first exchange cycles (i.e. smaller and shorter cycles) and the number of second exchange cycles (i.e. larger and longer cycles) are variable. In relation to the parameters of fill volume, dwell time, and glucose concentration as parameters or degrees of freedom of a peritoneal dialysis treatment regimen, the candidate set of peritoneal dialysis treatment regimens depicted in FIG. 9 corresponds to the candidate set depicted in FIG. 6.

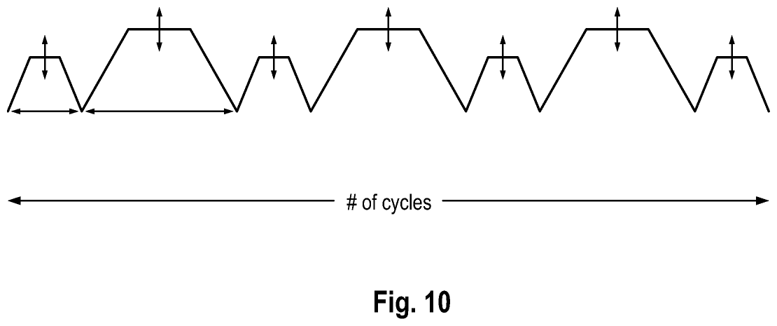

FIG. 10 is a graphical representation of another example candidate set of peritoneal dialysis treatment regimens to be simulated by the peritoneal dialysis treatment simulation. Similar to the candidate sets depicted in FIGS. 8 and 9, the first type of smaller and shorter exchange cycles alternate with the second type of larger and longer exchange cycles, and the number of first exchange cycles and the number of second exchange cycles are variable. In relation to the parameters of fill volume, dwell time and glucose concentration as parameters or degrees of freedom of a peritoneal dialysis treatment regimen, the candidate set of peritoneal dialysis treatment regimens depicted in FIG. 10 corresponds to the candidate set depicted in FIG. 7.

1-16. (canceled)

17. A method of generating a peritoneal dialysis treatment regimen, the method comprising: receiving a therapy target input;
receiving a patient characteristics input;
generating a plurality of peritoneal dialysis treatment regimens, each peritoneal dialysis treatment regimen comprising:
one or more first exchange cycles, each of the one or more first exchange cycles including a first inflow volume and a first dwell time; and
one or more second exchange cycles, each of the one or more second exchange cycles including a second inflow volume and a second dwell time,
wherein each peritoneal dialysis treatment regimen of the plurality of peritoneal dialysis treatment regimens comprises a number of first exchange cycles and a number of second exchange cycles, wherein at least one of the number of first exchange cycles and the number of second exchange cycles is variable;
simulating a peritoneal dialysis treatment outcome for each of the plurality of peritoneal dialysis treatment regimens based at least in part on the patient characteristics to generate a simulated treatment outcome; and
providing for selection a peritoneal dialysis treatment regimen to be applied for a specific treatment based on the simulated treatment outcome and the therapy target.

18. The method of claim 17, further comprising receiving an allowable treatment parameter range, wherein the plurality of peritoneal dialysis treatment regimens are generated based at least in part on the allowable treatment parameter range.

19. The method of claim 17, wherein a fixed ratio between the first dwell time and the second dwell time is predefined.

20. The method of claim 17, wherein a fixed ratio between the first inflow volume and the second inflow volume is predefined.

21. The method of claim 17, wherein each of the one or more first exchange cycles are shorter and smaller than each of the one or more second exchange cycles.

22. The method of claim 17, wherein a first glucose concentration is defined for the one or more first exchange cycles and a second glucose concentration is defined for the one or more second exchange cycles, wherein the first glucose concentration is larger than the second glucose concentration.

23. The method of claim 17, wherein the one or more first exchange cycles precede the one or more second exchange cycles.

24. The method of claim 17, wherein each of the one or more the first exchange cycles alternate with each of the one or more second exchange cycles.

25. The method of claim 17, wherein the number of first exchange cycles is independent of the number of second exchange cycles.

26. An apparatus for generating a peritoneal dialysis regimen, the apparatus comprising: a therapy target input unit; a patient characteristics input unit;
a peritoneal regimen generation unit configured to generate a plurality of peritoneal dialysis treatment regimens, each peritoneal dialysis treatment regimen comprising one or more first exchange cycles and one or more second exchange cycles,
wherein the one or more first exchange cycles comprise a first inflow volume and a first dwell time, and the one or more second exchange cycles comprise a second inflow volume and a second dwell time, and
wherein each peritoneal dialysis treatment regimen of the plurality of peritoneal dialysis treatment regimens comprises a number of first exchange cycles and a number of second exchange cycles, wherein at least one of the number of first exchange cycles and the number of second exchange cycles is variable;
a simulation unit configured to simulate a peritoneal dialysis treatment outcome for each peritoneal dialysis treatment regimen of the plurality of peritoneal dialysis treatment regimens to generate a simulated treatment outcome; and
a selection unit configured to facilitate selection of a peritoneal dialysis treatment regimen to be applied for a specific treatment, wherein the peritoneal dialysis treatment regimen to be applied is determined based on the simulated treatment outcome and the therapy target.

27. The apparatus of claim 26, wherein the apparatus is communicably coupled to a peritoneal dialysis machine.

28. The apparatus of claim 26, wherein the apparatus is communicably coupled to a database module of a patient database.

29. The apparatus of claim 26, wherein the number of first exchange cycles is independent of the number of second exchange cycles.

30. A peritoneal dialysis system, the system comprising:
a patient database comprising a database module; and
a peritoneal dialysis machine connected to the patient database, wherein the peritoneal dialysis machine is configured to receive a peritoneal dialysis treatment regimen from the patient database and operate in accordance with the received peritoneal dialysis treatment regimen, wherein the peritoneal dialysis treatment regimen received by the peritoneal dialysis machine is selected based on a simulated treatment outcome and a therapy target.

31. Computer-readable medium comprising instructions for the execution of a method according to claim 17 when the instructions are executed on a computer.
